(19) United States
(12) Patent Application Publication
(10) Pub. No.: US 2026/0090849 A1
(43) Pub. Date: Apr. 2, 2026

(54) IMAGE REGISTRATION METHOD AND DEVICE FOR SURGICAL ROBOT, ELECTRONIC DEVICE AND STORAGE MEDIUM

(71) Applicant: TINAVI MEDICAL TECHNOLOGIES CO., LTD., Beijing (CN)

(72) Inventors: Cheng ZHU, Beijing (CN); Junliang WANG, Beijing (CN); Tiechang LIU, Beijing (CN); Weijun ZHANG, Beijing (CN); Jie WANG, Beijing (CN); Lili JIANG, Beijing (CN)

(73) Assignee: TINAVI MEDICAL TECHNOLOGIES CO., LTD., Beijing (CN)

(21) Appl. No.: 19/410,738

(22) Filed: Dec. 5, 2025

Related U.S. Application Data
(63) Continuation of application No. PCT/CN2023/137406, filed on Dec. 8, 2023.

(30) Foreign Application Priority Data
Jun. 8, 2023 (CN) 202310678713.2

(51) Int. Cl. A61B 34/30; A61B 34/20; A61B 90/00; G06T 7/33; G06T 7/80; G06V 10/25; G06V 10/26; G06V 10/74; G16H 30/40

(52) U.S. Cl. CPC A61B 34/30; G06T 7/337; G06T 7/80; G06V 10/25; G06V 10/26; G06V 10/761; G16H 30/40; A61B 2034/2065; A61B 2090/376; G06T 2200/04; G06T 2207/10081; G06T 2207/10124; G06T 2207/30012; G06T 2207/30052; G06T 2207/30204; G06T 2207/30244; G06V 2201/033; G06V 2201/034

(57) ABSTRACT

An image registration method for a surgical robot, comprising: capturing a two-dimensional image of a surgical object and determining first guiding information in the two-dimensional image; acquiring a three-dimensional image of the surgical object and preoperative planning information, and determining second guiding information in the three-dimensional image; calculating first pose information according to the first guiding information and the second guiding information; adjusting the three-dimensional image according to the first pose information, and acquiring a digitally reconstructed two-dimensional image in the adjusted three-dimensional image; calculating the similarity between the two-dimensional image and the digitally reconstructed two-dimensional image; when the similarity meets a preset condition, projecting the preoperative planning information onto the two-dimensional image according to the first pose information; when the similarity does not meet the preset condition, updating the first pose information to obtain second pose information, and adjusting the three-dimensional image according to the second pose information.

### CROSS-REFERENCE TO RELATED APPLICATIONS

This application is a continuation of International Patent Application No. PCT/CN2023/137406 with a filing date of Dec. 8, 2023, designating the United States, now pending, and further claims priority to Chinese Patent Application No. 202310678713.2 with a filing date of Jun. 8, 2023. The content of the aforementioned applications, including any intervening amendments thereto, is incorporated herein by reference.

### TECHNICAL FIELD

The present disclosure relates to the technical field of image processing, and particularly relates to an image registration method and device for a surgical robot, as well as an electronic device and a storage medium.

### BACKGROUND ART

With the intensifying trend of population aging in China, the incidence of orthopedic diseases among the Chinese population has been gradually increasing. Spinal diseases account for a large proportion of these cases, and statistics show that more than 50% of people over 60 years old suffer from spinal disorders. Therefore, robot-assisted spinal navigation systems are of great significance.

At present, robot-assisted spinal navigation mainly includes the following methods: navigation imaging methods based on two-dimensional C-arm X-ray machines; intraoperative navigation imaging methods based on three-dimensional C-arm imaging; and navigation imaging methods based on preoperative three-dimensional computed tomography (CT) reconstruction and planning. Among these, navigation imaging methods based on two-dimensional C-arm X-ray machines are the most widely used, but they are not sufficiently intuitive and result in relatively low surgical efficiency. Intraoperative navigation imaging methods based on three-dimensional C-arm imaging have less ideal imaging quality, are expensive, and have low equipment penetration rates. Navigation imaging methods based on preoperative 3D CT reconstruction and planning cannot be accurately implemented without intraoperative spatial localization algorithms. Navigation imaging methods based on preoperative 3D CT reconstruction and planning combined with 2D/3D registration can avoid the above issues, and the main challenge lies in ensuring that the 2D/3D registration accuracy meets clinical requirements.

### SUMMARY OF THE INVENTION

In the prior art, the 2D/3D image registration methods used during surgical procedures lack an accurate calculation method for the initial pose of CT data, and the similarity calculation methods for CT image data and X-ray image data are also not sufficiently accurate, fast, or stable, resulting in low registration accuracy and low efficiency in 2D/3D image registration.

In view of this, in a first aspect, the present disclosure provides an image registration method for a surgical robot, comprising: capturing a two-dimensional image of a surgical object, and determining first guiding information in the two-dimensional image; acquiring a three-dimensional image of the surgical object and preoperative planning information, and determining second guiding information in the three-dimensional image; calculating first pose information according to the first guiding information and the second guiding information; adjusting the three-dimensional image according to the first pose information, and acquiring a digitally reconstructed two-dimensional image (digitally reconstructed radiograph) in the adjusted three-dimensional image; and calculating a similarity between the two-dimensional image and the digitally reconstructed two-dimensional image; where when the similarity meets a preset condition, projecting the preoperative planning information onto the two-dimensional image according to the first pose information; when the similarity does not meet the preset condition, updating the first pose information to obtain second pose information, and adjusting the three-dimensional image according to the second pose information.

According to some embodiments, the two-dimensional image comprises anteroposterior two-dimensional images and lateral two-dimensional images, the first guiding information comprises anteroposterior guiding information and lateral guiding information, and the capturing the two-dimensional images of the surgical object comprises: capturing an anteroposterior two-dimensional image of the surgical object at a first position, and determining the anteroposterior guiding information in the anteroposterior two-dimensional image; and capturing a lateral two-dimensional image of the surgical object at a second position, and determining the lateral guiding information in the lateral two-dimensional image; wherein the anteroposterior guiding information comprises an anteroposterior center position information of the surgical object in the anteroposterior two-dimensional image and anteroposterior naming information, and the lateral guiding information comprises a lateral center position information of the surgical object in the lateral two-dimensional image and lateral naming information.

According to some embodiments, the acquiring the three-dimensional image of the surgical object and determining the second guiding information in the three-dimensional image comprises: acquiring three-dimensional medical images of a patient's surgical region; performing ROI cropping on the three-dimensional medical images; performing single-segment segmentation and extraction on the cropped three-dimensional medical images; selecting the surgical object from at least one segment and checking the segmentation result; naming the surgical object; and calculating the center position of the surgical object by a deep learning algorithm, and taking information the naming and the center position of the surgical object as the second guiding information.

According to some embodiments, the two-dimensional image is captured by a two-dimensional image capturing device, and the calculating the first pose information according to the first guiding information and the second guiding information comprises: acquiring scale information in the two-dimensional image; acquiring internal parameters of the two-dimensional image capturing device; calculating external parameters of the two-dimensional image capturing device according to the scale information and the internal parameters; and obtaining the first pose information according to the external parameters and the first guiding information and the second guiding information.

According to some embodiments, the adjusting the three-dimensional image according to the first pose information and acquiring the digitally reconstructed two-dimensional image in the adjusted three-dimensional image comprises: processing the three-dimensional image, simulating an X-ray source, calculating image density values in the ray direction, and obtaining the digitally reconstructed two-dimensional image.

According to some embodiments, the calculating the similarity between the two-dimensional image and the digitally reconstructed two-dimensional image comprises: calculating the similarity using a normalized cross-correlation coefficient.

According to some embodiments, the calculating the similarity using a normalized cross-correlation coefficient comprises: calculating a mean value and a standard deviation of the two-dimensional image and the digitally reconstructed two-dimensional image; determining the normalized cross-correlation coefficient according to the mean value and the standard deviation; and determining the similarity according to the normalized cross-correlation coefficient.

According to some embodiments, formulas for calculating the mean value and the standard deviation of the two-dimensional image and the digitally reconstructed two-dimensional image are as follows:

$$μ ( K ) = \frac{1}{\vert Ω \vert} {\sum }_{p ∈ Ω} K ( p )$$

$$σ ( K ) = \sqrt{\frac{1}{\vert Ω \vert - 1} {\sum }_{p ∈ Ω} {( K ( p ) - μ ( K ) )}^{2}}$$

where K is the two-dimensional image or the digitally reconstructed two-dimensional image, μ is the mean value, σ is the standard deviation, Ω is a total number of image pixels, and p is a pixel size of an image corresponding to the two-dimensional image K or the digitally reconstructed two-dimensional image K.

According to some embodiments, a formula for determining the normalized cross-correlation coefficient (NCC) according to the mean value and standard deviation is as follows:

$${S}_{N C C} ( I , J , {c}_{r} , {c}_{c} , r ) = - \frac{1}{\vert {Ω}_{{c}_{r} , {c}_{c} , r} \vert} {\sum }_{p ∈ {Ω}_{{c}_{r} , {c}_{c} , r}} \frac{( I ( p ) - {μ}_{I} ) ( J ( p ) - {μ}_{J} )}{{σ}_{I} {σ}_{J}}$$

where I is the two-dimensional image, J is the digitally reconstructed two-dimensional image, cᵣ is a coordinate of a center position in the two-dimensional image, c_{c} is a coordinate of a center position in the digitally reconstructed two-dimensional image, r is half of a side length of the two-dimensional image and the digitally reconstructed two-dimensional image.

According to some embodiments, a formula for determining the similarity according to the normalized cross-correlation coefficient is as follows:

$${S}_{G N C C} ( I , J , {c}_{{r}_{1}} , {c}_{{c}_{1}} , {r}_{1} ) = {S}_{NCC} ( {∇}_{x} I , {∇}_{y} J , {c}_{{r}_{1}} , {c}_{{c}_{1}} , {r}_{1} ) + {S}_{NCC} ( {∇}_{x} I , {∇}_{y} J , {c}_{{r}_{1}} , {c}_{{c}_{1}} , {r}_{1} )$$

where ∇ₓI is an X-gradient image of the two-dimensional image, ∇_{y}I is a Y-gradient image of the two-dimensional image, ∇ₓJ is an X-gradient image of the two-dimensional image, ∇_{y}J is a Y-gradient image of the digitally reconstructed two-dimensional image, S_{GNCC} is a sum of the NCCs of X-gradient and Y-gradient gradient images, cᵣ_{₁} is a coordinate of a center position in the X-gradient images, c_{c}_{₁} is a coordinate of a center position in the Y-gradient images, r₁ is half of a side length of the X-gradient images and the Y-gradient images, and S_{GNCC}(I, J, Cᵣ_{₁}, c_{c}_{ᵢ}, r₁) is the similarity of the X-gradient images in a square region with cᵣ_{₁} as the center and 2r₁ as a side length and the Y-gradient images in a square region with cₑ, as the center and 2r₁ as a side length.

According to some embodiments, the projecting the preoperative planning information onto the two-dimensional image according to the first pose information comprises: projecting preoperatively planned pedicle screw positions onto the two-dimensional image.

According to some embodiments, the updating the first pose information to obtain the second pose information comprises: using the first pose information as an initial value, and updating the first pose information using a gradient-independent optimization algorithm to obtain the second pose information.

In a second aspect, the present disclosure further provides an image registration device for a surgical robot, comprising: a capturing assembly configured to capture two-dimensional images of a surgical object and determine first guiding information in the two-dimensional images; an acquisition assembly configured to acquire three-dimensional images of the surgical object and determine second guiding information in the three-dimensional images; an information processing assembly configured to:
calculate first pose information according to the first guiding information and the second guiding information; adjust the three-dimensional image according to the first pose information, and acquire a digitally reconstructed two-dimensional image in the adjusted three-dimensional image; calculate similarity between the two-dimensional image and the digitally reconstructed two-dimensional image; when the similarity meets a preset condition, project preoperative planning information onto the two-dimensional image according to the first pose information; when the similarity does not meet the preset condition, update the first pose information to obtain second pose information, and adjust the three-dimensional image according to the second pose information.

In a third aspect, the present disclosure further provides an electronic device, wherein it comprises: a processor; and a memory storing a computer program, which, when executed by the processor, causes the processor to perform the method according to the first aspect of the present disclosure.

In a fourth aspect, the present disclosure further provides a non-transitory computer-readable storage medium storing computer-readable instructions, wherein when executed by a processor, the instructions cause the processor to perform the method according to the first aspect of the present disclosure.

### Beneficial Effects

The image registration method for a surgical robot provided by the present disclosure extracts guiding information from intraoperative two-dimensional images and from preoperative three-dimensional images, registers the two-dimensional images with the three-dimensional images, and makes judgments based on the similarity between the two-dimensional images and the digitally reconstructed two-dimensional images. The algorithm has a fast iteration speed and high registration efficiency. Furthermore, by using the center position of the surgical object as guiding information and calculating the initial pose transformation matrix using the single-point method, the registration achieves high accuracy and speed, ensuring the smooth progress of surgical procedures (such as spinal surgery) and providing patients with a more comfortable surgical experience. This has significant implications for the clinical application of surgical robots. Verification shows that the image registration method provided by the present disclosure can achieve ten-thousand-layer registration within only five seconds, meeting clinical real-time requirements. Moreover, the image registration method provided by the present disclosure applies artificial intelligence deep learning algorithms for the extraction and segmentation of spinal segments, resulting in higher registration accuracy and efficiency, and an improved user experience.

### DETAILED DESCRIPTION OF THE DRAWINGS

In order to more clearly illustrate the technical solutions in the embodiments of the present application, the drawings used in the description of the embodiments are briefly introduced below. It is apparent that the drawings described below are only some embodiments of the present application, and those of ordinary skill in the art can obtain other drawings based on these drawings without departing from the scope of the present application.

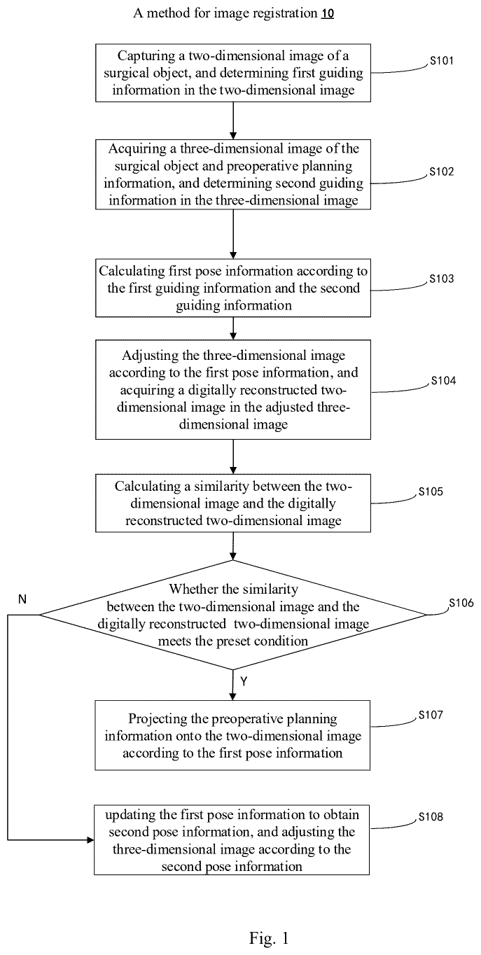

FIG. 1 illustrates an image registration method for a surgical robot provided by an embodiment of the present disclosure;

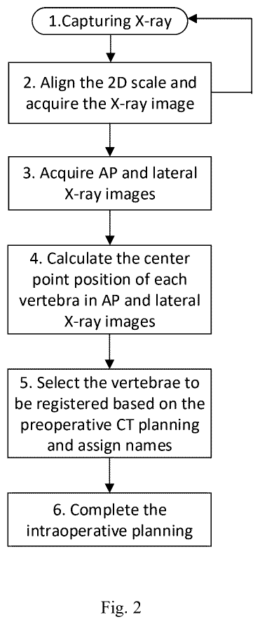

FIG. 2 illustrates intraoperative acquisition and planning of two-dimensional X-ray images in the image registration method for a surgical robot provided by an embodiment of the present disclosure;

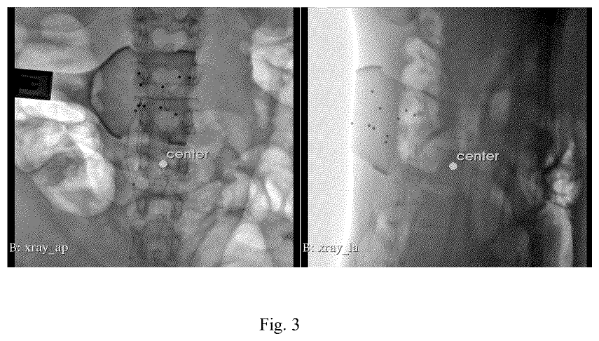

FIG. 3 illustrates calibration of the center position of the surgical object during intraoperative acquisition and planning of two-dimensional X-ray images in the image registration method for a surgical robot provided by an embodiment of the present disclosure;

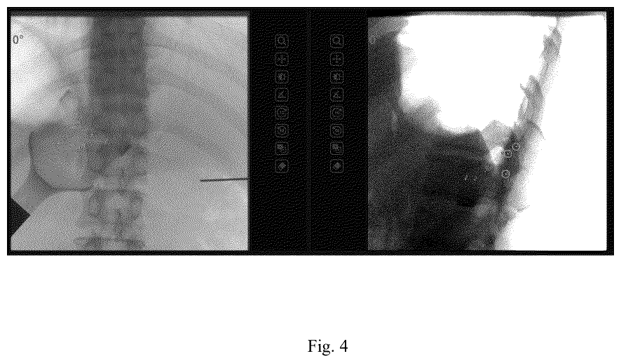

FIG. 4 illustrates detection of scale information in two-dimensional X-ray images during the registration process in the image registration method for a surgical robot provided by an embodiment of the present disclosure;

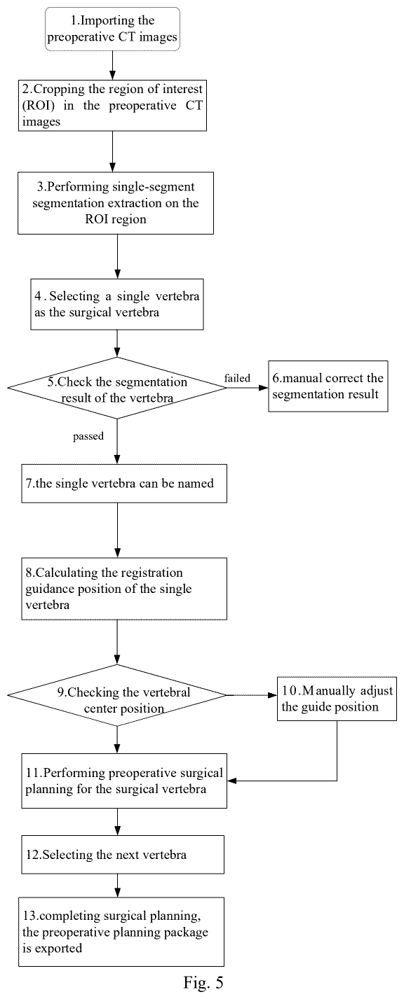

FIG. 5 illustrates preoperative CT data acquisition and surgical planning in the image registration method for a surgical robot provided by an embodiment of the present disclosure;

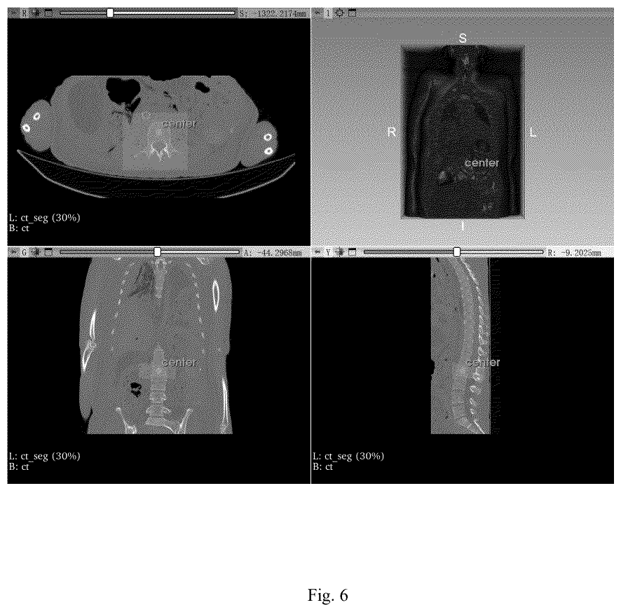

FIG. 6 illustrates calibration of the center position of the surgical object during preoperative CT data acquisition and surgical planning in the image registration method for a surgical robot provided by an embodiment of the present disclosure;

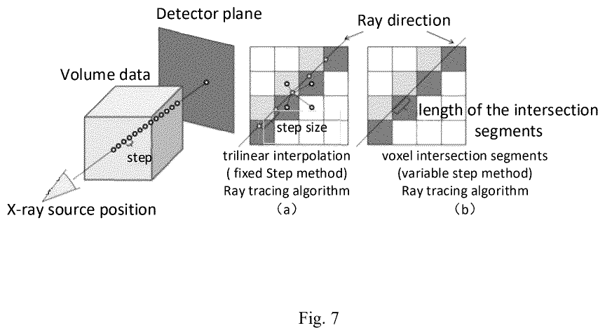

FIG. 7 illustrates the process of obtaining a digitally reconstructed two-dimensional image in the three-dimensional image according to the first pose information in the image registration method for a surgical robot provided by an embodiment of the present disclosure;

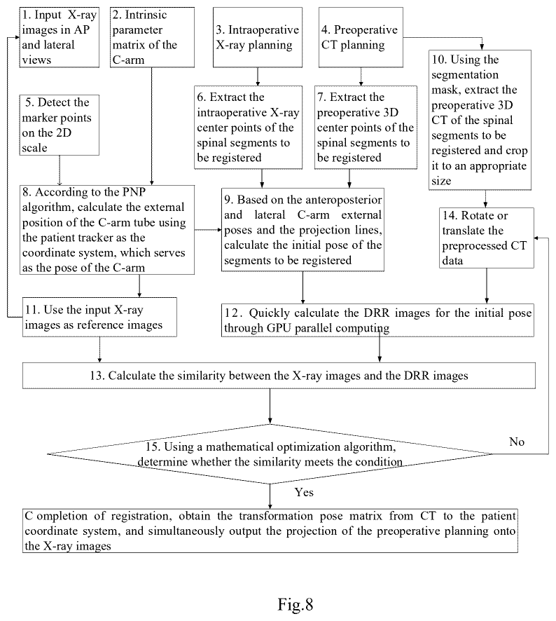

FIG. 8 illustrates an image registration method for a surgical robot provided by an embodiment of the present disclosure;

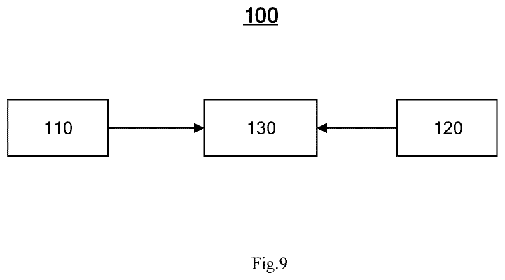

FIG. 9 illustrates an image registration device for a surgical robot provided by an embodiment of the present disclosure.

### DETAILED DESCRIPTION OF EMBODIMENTS

The technical solutions of the embodiments of the present application will be clearly and completely described below in conjunction with the accompanying drawings of the embodiments. It is apparent that the described embodiments are only part of the embodiments of the present application, and are not intended to limit the scope of all embodiments. Based on the embodiments disclosed in the present application, all other embodiments obtained by those skilled in the art without inventive efforts fall within the protection scope of the present application.

A preoperative-planning-based intraoperative 2D/3D registration imaging method requires accurate registration between intraoperatively acquired two-dimensional images (e.g., X-ray images) and preoperatively acquired three-dimensional images (e.g., CT images). The surgical robot performs surgery based on the registered images, or a robot-assisted surgical system uses the registered images for navigational assistance to aid manual surgery. In the 2D/3D image registration workflow, a series of specific algorithms are applied to determine factors such as rotation angles, translation displacements, image size, and correspondence between vertebrae in the images, thereby projecting the surgical planning onto the vertebra to be operated. In other words, images captured in the X-ray coordinate system are transformed to the preoperative CT coordinate system and then further transformed to the patient tracker coordinate system, establishing the relative spatial relationship between preoperative planning content (e.g., pedicle screw placement) and the vertebra to be operated on, meeting the clinical operational requirements of the surgical robot or robot-assisted surgical system.

The present disclosure provides an image registration method for a surgical robot for real-time intraoperative 2D/3D medical image registration. Using the registration algorithm of the present disclosure, preoperative three-dimensional CT data are spatially localized intraoperatively, and preoperative surgical planning (e.g., the specific placement of pedicle screws on vertebrae to be operated) is incorporated. This enables intraoperative navigation to be displayed in 3D rather than 2D, enhancing the physician's experience and improving surgical efficiency. Experimental validation shows that the image registration method of the present disclosure can achieve registration across thousands of slices within only 5 seconds, satisfying clinical real-time requirements, with high registration accuracy and efficiency and improved user experience.

According to an embodiment of the present disclosure, as shown in FIG. 1, the image registration method 10 for a surgical robot includes steps S101 through S108.

In step S101, two-dimensional images of the surgical object are captured, and first guidance information in the two-dimensional images is determined.

The surgical object may be a target object requiring surgical operation. For example, in minimally invasive spinal surgery, the surgical object may include one or more vertebrae to be operated on.

Specifically, intraoperative real-time two-dimensional images of the surgical object (e.g., X-ray images) are captured, and the images are localized to satisfy subsequent 2D/3D registration requirements. The first guidance information in the two-dimensional images includes vertebral naming information, vertebral center position information, and may further include vertebral edge positions, vertebral contour information, and two-dimensional scale information.

For example, the vertebral naming information of the vertebra to be operated may include lumbar L1, thoracic T1, sacral S1, etc. The vertebral center position information may include the coordinates of the center point located on the midline of the vertebra in two dimensions.

In step S102, three-dimensional images of the surgical object and preoperative planning information are acquired, and second guidance information in the three-dimensional images is determined.

For example, the three-dimensional image of the surgical object is obtained preoperatively using CT imaging equipment, and a preoperative localization operation is performed. The second guidance information in the three-dimensional image includes vertebral naming information and vertebral center position information, and may additionally include vertebral edge positions and vertebral contour information.

Exemplarily, the vertebral naming information in the three-dimensional image of the vertebra to be operated may be lumbar L1, thoracic T1, sacral S1, etc., and the vertebral center position information may include the coordinates of the center point on the midline of the vertebra.

In step S102, acquiring preoperative planning information of the surgical object may include receiving surgical planning information input by a user regarding the vertebra to be operated. For example, the user may select an appropriate surgical plan according to the patient's condition; for a patient with previously implanted hardware, the planning may include the placement of pedicle screws and determination of their positions.

In step S103, first pose information is calculated based on the first guidance information and the second guidance information.

For example, first pose information is calculated based on the first guidance information in the two-dimensional image (e.g., vertebral naming information, vertebral center position) and the second guidance information in the three-dimensional image (e.g., vertebral naming information, vertebral center position). The first pose information includes an initial pose transformation matrix for converting from the CT image coordinate system to the patient tracker coordinate system.

Optionally, the first pose information is calculated using a one-point method to obtain the initial pose transformation matrix of the three-dimensional image.

In step S104, the three-dimensional image is adjusted according to the first pose information, and a digitally reconstructed two-dimensional image (digitally reconstructed radiograph) is obtained from the adjusted three-dimensional image.

For example, the three-dimensional image is transformed according to the initial pose transformation matrix, and its projection onto the plane of the two-dimensional image is obtained, generating the digitally reconstructed radiographs (DRR) corresponding to the three-dimensional image.

Optionally, the projection onto the two-dimensional image plane may be computed using a simulated light source algorithm. The adjustment includes angle rotation and/or position translation of the three-dimensional image according to the first pose information—for example, applying the rotation matrix in the first pose information to rotate the three-dimensional image, and applying the translation matrix in the first pose information to translate the three-dimensional image.

In step S105, the similarity between the two-dimensional image and the digitally reconstructed two-dimensional image is calculated.

For example, the similarity between the digitally reconstructed two-dimensional image generated by the algorithm and the intraoperatively acquired two-dimensional image is computed, which may be calculated using a normalized cross-correlation coefficient.

In step S106, it is determined whether the similarity satisfies a preset condition. When the similarity satisfies the preset condition, step S107 is executed; when the similarity does not satisfy the preset condition, step S108 is executed.

For example, the similarity between the two-dimensional image and the digitally reconstructed two-dimensional image is compared, and the degree of similarity is judged according to a preset similarity criterion. When the similarity satisfies the preset condition, the two images are considered highly similar and the registration is successful. When the similarity does not satisfy the preset condition, the registration is considered inaccurate, and the three-dimensional image needs to be further adjusted and re-registered.

In step S107, when the similarity satisfies the preset condition, the preoperative planning information is projected onto the two-dimensional image based on the first pose information.

For example, when the similarity between the two-dimensional image and the digitally reconstructed two-dimensional image meets a preset condition, the two images are considered successfully registered. Based on the first pose information, the preoperative planning information is projected onto the two-dimensional image, and the surgical robot or robot-assisted surgical system performs surgery or assists manual surgery according to the positions of the preoperative planning information on the two-dimensional image.

In step S108, when the similarity does not meet the preset condition, the first pose information is updated to obtain the second pose information, and the three-dimensional image is adjusted according to the second pose information.

For example, when the similarity between the two-dimensional image and the digitally reconstructed two-dimensional image does not satisfy the preset condition, the image registration is unsuccessful, and the three-dimensional image needs to be further adjusted and re-registered.

Optionally, based on the first pose information, the initial pose transformation matrix is used as the initial value, and the parameters of the initial pose transformation matrix are updated to obtain the second pose information.

Optionally, the initial pose transformation matrix includes a rotation matrix and a translation matrix. By iteratively updating the rotation matrix and/or the translation matrix parameters along six degrees of freedom (6 DOF), the second pose information is obtained. The three-dimensional image is then adjusted according to the second pose information.

The steps S104 to S108 are repeated iteratively, updating the pose transformation matrix multiple times, adjusting the three-dimensional image multiple times, obtaining the digitally reconstructed two-dimensional image from the adjusted three-dimensional image, and calculating the similarity between the two-dimensional image and the digitally reconstructed two-dimensional image until the similarity meets the preset condition. In this way, intraoperative image registration is completed, the preoperative planning information is projected onto the two-dimensional image, and the intraoperative real-time navigation system is activated.

The image registration method for a surgical robot provided by the above embodiments of the present disclosure extracts guidance information from intraoperative two-dimensional images and preoperative three-dimensional images, performs registration between the two-dimensional image and the three-dimensional image, and determines registration success based on the similarity between the two-dimensional image and the digitally reconstructed two-dimensional image. This method achieves fast iterative convergence and high registration efficiency.

According to an embodiment of the present disclosure, in step S101 of the image registration method 10, the two-dimensional images include anteroposterior (AP) and lateral images. The first guidance information includes AP guidance information and lateral guidance information. Capturing the two-dimensional images of the surgical object comprises:

Capturing the AP two-dimensional image of the surgical object at a first position and determining the AP guidance information in the AP image. The AP guidance information includes the AP center position information and AP naming information of the surgical object in the AP image.

For example, the first position includes the front view of the surgical object (such as the vertebra to be operated on), and the AP two-dimensional image is captured from the front of the surgical object. Exemplarily, in spinal surgery, the AP naming information in the AP image can be the vertebral names, such as lumbar L1, thoracic T1, sacral S1, etc. The AP center position information can be the coordinates of the center point located on the midline of the vertebra in two dimensions.

Capturing the lateral two-dimensional image of the surgical object at a second position and determining the lateral guidance information in the lateral image. The lateral guidance information includes the lateral center position information and lateral naming information of the surgical object in the lateral image.

For example, the second position includes the lateral view of the surgical object (such as the vertebra to be operated on), and the lateral two-dimensional image is captured from the side of the surgical object. Exemplarily, in spinal surgery, the lateral naming information in the lateral image can be the vertebral names, such as lumbar L1, thoracic T1, sacral S1, etc. The lateral center position information can be the coordinates of the center point located on the vertebral midline in two dimensions.

The imaging projection lines of the first position and the second position can be orthogonal. Orthogonal projection lines for the AP and lateral views facilitate subsequent registration with the center positions of the three-dimensional image. According to an embodiment, the AP and lateral guidance information are obtained via a deep learning algorithm and verified manually.

For example, the AP two-dimensional image of the vertebra to be operated on is obtained via the X-ray machine. The AP guidance information, such as the AP vertebral center position, is extracted using a deep learning algorithm, e.g., by applying an AABB bounding box algorithm to crop the image region in the AP image. Similarly, the lateral guidance information, such as the lateral vertebral center position, is obtained from the lateral image.

According to an embodiment of the present disclosure, when capturing the AP and lateral two-dimensional images of the surgical object at the first and second positions, a scale (e.g., steel ball markers) is placed so that the AP and lateral images contain scale information, which is used to calculate the external parameters of the two-dimensional imaging device.

Exemplarily, the first guidance information of the two-dimensional image of the surgical object can be obtained intraoperatively through X-ray imaging and planning.

According to an embodiment, as shown in FIG. 2, intraoperative X-ray imaging and planning includes:

Capturing X-ray images of the patient's surgical region intraoperatively.

Determining the position of a two-dimensional scale so that it is clearly visible in the X-ray image, obtaining at least two X-ray images of the surgical object in the AP and lateral views.

Computing the u-v coordinates of the target vertebra (the surgical target) in the anteroposterior (AP) X-ray image and the lateral X-ray image-specifically, the u-v coordinates of the vertebral center point located at the central position along the vertebral midline in each image. Then, manually verify the accuracy of the AP and lateral vertebral center locations, as shown in FIG. 3. The u-v values of the AP and lateral vertebral centers are typically calculated using deep-learning-based methods.

Determining the AP vertebral center information and lateral vertebral center information based on the (u, v) values, and naming the vertebra in the AP and lateral views to obtain the AP naming information and lateral naming information, thereby obtaining the first guidance information in the two-dimensional images.

Through intraoperative X-ray imaging and corresponding planning as described above, the required two-dimensional images of the surgical object are obtained, and the surgical information of the surgical object (e.g., vertebral center position and vertebral name) is determined in the two-dimensional images, which constitutes the first guidance information.

Exemplarily, the first guidance information of the surgical object (such as vertebral center position and vertebral name) can be automatically calculated by the image registration system or manually obtained through the software interface of the system.

According to an embodiment of the present disclosure, in step S102 of the image registration method 10 for a surgical robot provided by the present disclosure, the three-dimensional image of the surgical object is acquired preoperatively. Acquiring the three-dimensional image of the surgical object and determining the second guidance information in the three-dimensional image comprises:

Acquiring a three-dimensional medical image of the patient's surgical region.

Cropping the region of interest (ROI) in the three-dimensional medical image.

Performing single-segment segmentation extraction on the cropped three-dimensional medical image.

Selecting the surgical object from at least one segment and checking the segmentation results.

Naming the surgical object.

Calculating the center position of the surgical object using a deep learning algorithm, and using information of the naming and center position of the surgical object as the second guidance information.

According to an exemplary embodiment, the second guidance information of the three-dimensional image of the surgical object can be determined preoperatively based on CT images and preoperative surgical planning. As shown in FIG. 5, the specific steps for preoperative planning based on CT images include:

Importing the preoperative CT images of the patient's surgical region.

Cropping the region of interest (ROI) in the preoperative CT images. This reduces the visualization range of the CT images, allowing the user to focus on the ROI and improving user experience. Additionally, the reduced computation range enhances algorithm efficiency. Exemplarily, the ROI can be set according to user requirements.

Performing single-segment segmentation extraction on the ROI region.

Optionally, during single-segment segmentation extraction, a deep learning-based algorithm, semi-automatic traditional algorithm, or manual correction can be used to remove irrelevant adjacent vertebral segments, ensuring that subsequent algorithms operate on a single vertebral segment without interference. The present disclosure is not limited in this regard.

Selecting a single vertebra as the surgical vertebra and checking the segmentation result. If the segmentation is unsatisfactory, manual correction can be performed.

Performing anatomical structure identification and naming of the surgical vertebra. For example, the surgical vertebra can be named according to its anatomical position, such as lumbar L1, thoracic T1, sacral S1, etc.

Calculating the registration guidance position of the single vertebra. The registration guidance position is the vertebral center of the surgical vertebra, which is used to calculate the initial pose during registration.

Optionally, the vertebral center of the surgical vertebra can be determined using a deep learning algorithm. For example, an AABB bounding box algorithm can be applied to crop the segmentation region until the vertebral center position, as shown in FIG. 6, is identified.

Checking and calibrating the vertebral center position calculated by the algorithm. The vertebral center position should remain near the vertebral midline. If there is significant deviation, manual adjustment can be applied to correct the registration guidance position.

Exemplarily, a deviation threshold for the vertebral center position can be set according to user requirements. When the deviation exceeds the preset threshold, the user can manually adjust the vertebral center to the desired position.

Performing preoperative surgical planning for the surgical vertebra. For example, depending on the patient's condition, appropriate surgical procedures can be chosen. For patients requiring pedicle screw placement, the position of pedicle screws can be planned preoperatively.

Completing preoperative planning for the current surgical vertebra, selecting the next vertebra, and returning to step 4.

Exemplarily, the user inputs information of the surgical vertebrae, including the number and positions of the vertebrae. After completing segmentation and preoperative planning for one vertebra, the next vertebra is automatically selected for segmentation and planning, until all surgical vertebrae are segmented and planned, after which step 10 is performed.

After completing surgical planning for all surgical vertebrae, the preoperative planning package is exported.

According to an embodiment of the present disclosure, in step S103 of the image registration method 10 for a surgical robot, the two-dimensional image is captured by a two-dimensional imaging device (such as a C-arm or X-ray machine). Calculating the first pose information based on the first guidance information and the second guidance information comprises:

Acquiring the scale information from the two-dimensional image.

For example, as shown in FIG. 4, detecting markers on a 2D scale in the X-ray image. The steel ball positions on the X-ray image can be used as marker points, and their positions are obtained using a pre-set algorithm, which can be a deep learning-based algorithm.

Acquiring the internal parameters of the two-dimensional imaging device.

The two-dimensional imaging device includes a C-arm, whose internal parameters can be represented by the C-arm intrinsic matrix. For example, the intrinsic matrix of the C-arm is:

$${M}_{i n} = [ \begin{matrix}f / {d}_{x} & 0 & {u}_{0} \\ 0 & f / {d}_{y} & {v}_{0} \\ 0 & 0 & 1\end{matrix} ] = [ \begin{matrix}{f}_{x} & 0 & {u}_{0} \\ 0 & {f}_{y} & {v}_{0} \\ 0 & 0 & 1\end{matrix} ]$$

where Mᵢₙ is the intrinsic matrix of the C-arm, f is the focal length, dₓ and d_{y} are the horizontal and vertical pixel spacing of the two-dimensional image, and u₀, v₀ are the horizontal and vertical coordinates of the vertebral center in the two-dimensional image.

Exemplarily, the vertebral center positions in the first guidance information can be (u₁,v₁),(u₂,v₂), etc.

Calculating the external parameters of the two-dimensional imaging device based on the scale information and the internal parameters.

According to an embodiment of the present disclosure, the PNP (Perspective-n-Point) algorithm is used with the patient tracker coordinate system to calculate the external parameters of the C-arm gantry, which serves as the pose of the C-arm.

The PNP algorithm solves the correspondence between 3D points and 2D points. Using n pairs of 3D points and corresponding 2D projection points, the external parameters (pose) of the camera can be solved by minimizing the re-projection error, even if the camera intrinsics are known or unknown. For example, the C-arm gantry external parameters can be obtained in the patient tracker coordinate system via the PNP algorithm.

Obtaining the first pose information based on the external parameters, the first guidance information, and the second guidance information.

For example, based on the determined external parameters of the C-arm and the projection lines corresponding to the vertebral centers in the AP and lateral X-ray images, the initial pose of the vertebra to be registered, namely the initial pose transformation matrix is calculated.

The initial pose transformation matrix is calculated using an initial pose algorithm, which provides a coarse 2D/3D registration result.

Exemplarily, the initial pose algorithm uses the one-point method. The procedure for determining the initial pose transformation matrix is as follows:

Determine the transformation matrix between the C-arm center coordinate system and the CT coordinate system based on patient positioning during X-ray acquisition, corresponding to intraoperative position coding. Combine the AP and lateral external parameters to obtain the rotation matrix R of the initial pose matrix Mᵢₙᵢₜ from the patient tracker coordinate system to the CT coordinate system. Then, based on the projection lines corresponding to the intraoperative X-ray center point in the AP and lateral views, obtain the midpoint P_{CPAT} of the perpendicular bisector of the two projection lines in the patient-tracker coordinate system, as well as the preoperative CT center P_{CCT}. Combining with the rotation matrix, the translation component T of the initial pose matrix is obtained. The initial pose transformation matrix is:

$${M}_{init} = [ \begin{matrix}R & T\end{matrix} ] R * {Pc}_{P A T} + T = {Pc}_{C T} = > T = {Pc}_{C T} - R * {Pc}_{P A T} \qquad ( 1 )$$

where Mᵢₙᵢₜ is the initial pose matrix, R is the rotation part, T is the translation part, P_{CPAT} is the midpoint of the perpendicular bisector of the two projection lines, and P_{CCT} is the preoperative CT center.

According to an embodiment of the present disclosure, as shown in formula (1), the first pose information includes rotational and translational components and has six degrees of freedom (6 DOF).

The above embodiments of the present disclosure use the vertebral center position as guidance information and calculate the initial pose transformation matrix using the one-point method. This ensures high registration accuracy and speed, facilitating smooth surgical procedures (e.g., spinal surgery) and providing patients with a better surgical experience. It is of great significance for the clinical application of surgical robots.

According to an embodiment of the present disclosure, in step S104 of the image registration method 10 for a surgical robot, adjusting the three-dimensional image based on the first pose information and obtaining the digitally reconstructed two-dimensional image from the adjusted three-dimensional image comprises:

Using a GPU (Graphics Processing Unit) to process the three-dimensional image, simulating an X-ray source, and calculating image density values along the ray direction to obtain the digitally reconstructed two-dimensional image.

The generation of DRR (Digitally Reconstructed Radiographs) is a key step in density-based 2D/3D registration algorithms. Since the DRR generation algorithm is called multiple times during registration, improving its computational speed is highly important.

As shown in FIG. 7, in the process of obtaining digitally reconstructed radiographs (DRR) from the adjusted three-dimensional images, the method used for 2D/3D registration is based on the line integral approach, which can simulate X-ray imaging. There are two main computational methods for generating DRR by line integration. One is the fixed-step method based on trilinear interpolation (as shown in FIG. 7(a)), where density values are calculated at sampling points with a fixed step along the ray direction. The other is the variable-step method based on voxel intersection segments (as shown in FIG. 7(b)), where density values are calculated along the intersection segments, and this method is slower but more accurate. Both types of algorithms are suitable for GPU-accelerated computation.

According to an embodiment of the present disclosure, in step S105 of the image registration method 10 for a surgical robot provided by the present disclosure, calculating the similarity between the two-dimensional image and the digitally reconstructed two-dimensional image comprises:

Calculating the similarity using the normalized cross-correlation coefficient (NCC).

The similarity between the X-ray image and the DRR refers to the degree of similarity between the real X-ray image and the algorithm-generated DRR image. In this embodiment, the normalized cross-correlation coefficient (NCC) is used. The normalized cross-correlation method is a commonly used cost function and exhibits good robustness to scaling and translation of distributions.

According to an embodiment of the present disclosure, in step S105 of the image registration method 10 for a surgical robot, using the normalized cross-correlation coefficient to calculate the similarity comprises:

Calculating the mean and standard deviation of the two-dimensional image and the digitally reconstructed two-dimensional image.

For example, the method first calculates the mean and standard deviation of the two-dimensional image (X-ray image) and the digitally reconstructed two-dimensional image.

The mean and standard deviation of the two-dimensional image K or the digitally reconstructed two-dimensional image K are defined as follows:

$$μ ( K ) = \frac{1}{\vert Ω \vert} {\sum }_{p ∈ Ω} K ( p ) \qquad ( 2 )$$

$$σ ( K ) = \sqrt{\frac{1}{\vert Ω \vert - 1} {\sum }_{p ∈ Ω} {( K ( p ) - μ ( K ) )}^{2}} \qquad ( 3 )$$

where μ is the mean, σ is the standard deviation, Ω is the total number of pixels, and p is the pixel of the two-dimensional image K or the digitally reconstructed two-dimensional image K.

The normalized cross-correlation coefficient is then determined according to the mean and standard deviation. NCC is calculated as:

$${S}_{N C C} ( I , J , {c}_{r} , {c}_{c} , r ) = - \frac{1}{\vert {Ω}_{{c}_{r} , {c}_{c} , r} \vert} {\sum }_{p ∈ {Ω}_{{c}_{r} , {c}_{c} , r}} \frac{( I ( p ) - {μ}_{I} ) ( J ( p ) - {μ}_{J} )}{{σ}_{I} {σ}_{J}} \qquad ( 4 )$$

where I is the two-dimensional image, J is the digitally reconstructed two-dimensional image, cᵣ is a coordinate of a center position in the two-dimensional image, c_{c} is a coordinate of a center position in the digitally reconstructed two-dimensional image, r is half of a side length of the two-dimensional image and the digitally reconstructed two-dimensional image.

The similarity is then determined according to the normalized cross-correlation coefficient.

Meanwhile, to make the registration process more stable and to account for the differences in value ranges between the X-ray image and the DRR, in this embodiment, the X and Y gradients of the two images are first calculated using the Sobel operator. Then the NCC of the X-gradient and Y-gradient images is computed, and the sum of the two NCCs is taken as the image similarity. The formula is as follows, where S_{GNCC} is the NCC of the gradient images:

$${S}_{G N C C} ( I , J , {c}_{{r}_{1}} , {c}_{{c}_{1}} , {r}_{1} ) = {S}_{NCC} ( {∇}_{x} I , {∇}_{y} J , {c}_{{r}_{1}} , {c}_{{c}_{1}} , {r}_{1} ) + {S}_{NCC} ( {∇}_{x} I , {∇}_{y} J , {c}_{{r}_{1}} , {c}_{{c}_{1}} , {r}_{1} ) \qquad ( 5 )$$

where ∇ₓI is an X-gradient image of the two-dimensional image, ∇_{y}I is a Y-gradient image of the two-dimensional image, ∇ₓJ is an X-gradient image of the two-dimensional image, ∇_{y}J is a Y-gradient image of the digitally reconstructed two-dimensional image, S_{GNCC} is a sum of the NCCs of X-gradient and Y-gradient gradient images, Cᵣ_{₁} is a coordinate of a center position in the X-gradient images, c_{c}_{₁} is a coordinate of a center position in the Y-gradient images, r₁ is half of a side length of the X-gradient images and the Y-gradient images, and S_{GNCC}(I, J, cᵣ_{₁}, C_{c}_{₁}, r₁) is the similarity of the X-gradient images in a square region with cᵣ_{₁} as the center and 2r₁ as a side length and the Y-gradient images in a square region with c_{c}_{₁} as the center and 2r₁ as a side length.

To prevent the result from deviating too far from the initial value and causing unpredictable extreme cases, in this embodiment, the regularization function is defined as follows, where σ is the variance of the translation or rotation. The translation range can be between 30 mm and 70 mm, and the rotation range can be between 30° and 80°.

Exemplarily, in this embodiment, the parameters are set as a translation of 50 mm and a rotation of 60°.

$$log ( \sqrt{2 {σ}^{2} π} ) - log ( Folded - Norm ( x ) ) \qquad ( 6 )$$

where Folded-Norm refers to the normal distribution of translation or rotation:

$${f}_{Y} ( x ; μ , {σ}^{2} ) = \frac{1}{\sqrt{2 π {σ}^{2}}} {e}^{- \frac{{( x - μ )}^{2}}{2 {σ}^{2}}} + \frac{1}{\sqrt{2 π {σ}^{2}}} {e}^{- \frac{{( x + μ )}^{2}}{2 {σ}^{2}}} \qquad ( 7 )$$

This formula ensures that the regularization function becomes large when the independent variable exceeds the variance.

Finally, the similarity formula is as follows:

$${min}_{θ ∈ SE ( 3 )} λ S ( 𝒫 ( I ; θ ) , J ) + ( 1 - λ ) ( θ ) \qquad ( 8 )$$

where SE(3) is the three-dimensional special Euclidean group for rigid body motion (rotation and translation), θ is the pose change in Euclidean space, S is the similarity calculation formula, R is a function of the pose change magnitude θ in Euclidean space, and λ is an adjustable hyperparameter, typically set to a weight of 0.1.

According to an embodiment of the present disclosure, in step S107 of the image registration method 10 for a surgical robot provided by the present disclosure, projecting the preoperative planning information onto the two-dimensional image according to the first pose information comprises:

Projecting the preoperatively planned pedicle screw positions onto the two-dimensional image.

For example, upon completion of registration, the transformation pose information from the CT coordinate system to the patient tracker coordinate system is obtained, while simultaneously outputting the projection of the preoperative planning onto the X-ray images.

According to an embodiment of the present disclosure, in step S108 of the image registration method 10 for a surgical robot provided by the present disclosure, updating the first pose information to obtain the second pose information comprises:

Using the first pose information as an initial value, and updating the first pose information by adopting a gradient-independent optimization algorithm to obtain the second pose information.

Exemplarily, the preset condition may be a preset similarity value set by the user according to requirements. After calculating the similarity between the two-dimensional image and the digitally reconstructed two-dimensional image in step S105, the value of the similarity is compared with the preset value to determine the degree of similarity. When the similarity between the two-dimensional image and the digitally reconstructed two-dimensional image meets the preset condition, it indicates that the two images are highly similar, and registration can be considered successful. When the similarity between the two-dimensional image and the digitally reconstructed two-dimensional image does not meet the preset condition, it indicates that image registration has a deviation, and the three-dimensional image needs to be further adjusted, and registration is performed again.

When the similarity between the two-dimensional image and the digitally reconstructed two-dimensional image does not meet the preset condition, the first pose information is used as the initial value, and the first pose information is updated using a gradient-independent optimization algorithm to obtain the second pose information.

Through the above gradient-independent mathematical optimization algorithm, it can be determined whether the similarity meets the preset condition. If the similarity does not meet the preset condition, the pose of the CT data is changed, and iterative optimization is performed until the preset condition is satisfied, so that image registration can be performed based on the first pose information or the updated second pose information.

Exemplarily, image registration based on the first pose information or the updated second pose information may include using a multi-stage, multi-resolution pyramid hierarchical registration method. In this embodiment, a two-stage method is used, that is, first using a low-resolution image with a large-area search optimizer algorithm to find the optimal value; then using a high-resolution image with a fast optimizer algorithm.

In the first stage, a fixed resolution image of 256×256 is selected, and the Covariance Matrix Adaptation Evolution Strategy (CMA-ES) optimizer is selected. The advantage of this optimizer is that it does not require calculating the gradient of the loss function, and it achieves good optimization results. It is also one of the modern optimizers least sensitive to the initial value. However, this optimizer requires a large number of loss function calculations in each iteration, making it slow. The CMA-ES algorithm requires the input of the initial value of the function to be optimized, the optimization parameter sequence, and the variance of each parameter.

In this embodiment, the initial value used is the initial pose transformation matrix from the patient tracker coordinate system to the CT coordinate system obtained by the PNP algorithm solving the anteroposterior and lateral external parameters. The optimization parameter list includes 6 degrees of freedom (6 parameters) of translation and rotation of the spinal segments to be registered; the translation variances in XYZ are all 5 mm, and the three rotation variances are 15 degrees, with 100 samples taken for each CMA-ES iteration.

In the second stage, in this embodiment, the image size after ½ downsampling is used as the registration optimization image. If this resolution is less than 256, the original X-ray image size is directly used. At this time, the slower evolutionary algorithm cannot be used as the optimizer. However, for convenience of solving, a gradient-independent optimization algorithm is still required. For example, BOBYQA and NEWUOA are optimizers that do not require a large number of loss function calculations per iteration and can be used.

According to an embodiment of the present disclosure, in the image registration method 10 for a surgical robot provided by the present disclosure, the two-dimensional image capturing device includes an X-ray machine, the X-ray machine is integrated with the surgical robot, and the surgical object includes the spinal vertebrae to be operated on.

The complete intraoperative registration process is described below in conjunction with specific embodiments.

According to an embodiment of the present disclosure, as shown in FIG. 8, real-time registration between intraoperative X-ray images and preoperative CT image data during surgery includes the following steps:
Step 1: Input two X-ray images in the anteroposterior and lateral views.
Step 2: Input the intrinsic parameter matrix of the C-arm (i.e., the X-ray image capturing device),

$${M}_{i n} = [ \begin{matrix}f / {d}_{x} & 0 & {u}_{0} \\ 0 & f / {d}_{y} & {v}_{0} \\ 0 & 0 & 1\end{matrix} ] = [ \begin{matrix}{f}_{x} & 0 & {u}_{0} \\ 0 & {f}_{y} & {v}_{0} \\ 0 & 0 & 1\end{matrix} ] \qquad ( 9 )$$

where Min is the intrinsic parameter matrix of the C-arm, f is the focal length of the C-arm, dₓ is the horizontal spacing of the pixels in the two-dimensional image, d_{y} is the vertical spacing of the pixels in the two-dimensional image, u₀ is the horizontal coordinate of the vertebral center in the two-dimensional image, and v₀ is the vertical coordinate of the vertebral center in the two-dimensional image.
Step 3: Load intraoperative X-ray planning, including the naming fields of the spinal segments to be registered in the anteroposterior and lateral views and the guiding positions for 2D X-ray registration.
Step 4: Import the preoperative CT planning package, including the naming fields of the spinal segments to be registered and the 3D CT guiding positions for each segment.
Step 5: Detect the marker points on the 2D scale: use the positions of the steel beads in the X-ray scale as marker points and determine their positions through an algorithm.
Step 6: From the intraoperative planning, extract the intraoperative X-ray center points of the spinal segments to be registered and record them as (u₁,v₁),(u₂,v₂).
Step 7: From the preoperative CT planning package, extract the preoperative 3D center points of the spinal segments to be registered and record them as (x₀, y₀, z₀).
Step 8: According to the PNP algorithm, calculate the external position of the C-arm tube using the patient tracker as the coordinate system, which serves as the pose of the C-arm.
Step 9: Based on the anteroposterior and lateral C-arm external poses and the projection lines, calculate the initial pose of the segments to be registered. The initial pose algorithm is used to calculate a coarse registration result for 2D/3D registration, for example, using the single-point method to calculate the initial pose.
Step 10: Using the segmentation mask, extract the preoperative 3D CT of the spinal segments to be registered and crop it to an appropriate size.
Step 11: Use the input X-ray images as reference images.
Step 12: Quickly calculate the DRR images for the initial pose through GPU parallel computing.
Step 13: Calculate the similarity between the X-ray images and the DRR images.
Step 14: Using a mathematical optimization algorithm, determine whether the similarity meets the condition. If not, preprocessed CT data are rotated or translated to change the pose of the CT data, and the process is iteratively optimized until the termination condition is met.
Step 15: Upon completion of registration, obtain the transformation pose matrix from CT to the patient coordinate system, and simultaneously output the projection of the preoperative planning onto the X-ray images.

The image registration method for a surgical robot provided in one or more embodiments of the present disclosure extracts guiding information from intraoperative two-dimensional images and from preoperative three-dimensional images, registers the two-dimensional images with the three-dimensional images, and makes judgments based on the similarity between the two-dimensional images and the digitally reconstructed two-dimensional images. The algorithm has a fast iteration speed and high registration efficiency.

Furthermore, by using the center position of the surgical object as guiding information and calculating the initial pose transformation matrix using the single-point method, the registration achieves high accuracy and speed, ensuring the smooth progress of surgical procedures (such as spinal surgery) and providing patients with a more comfortable surgical experience. This has significant implications for the clinical application of surgical robots. Verification shows that the image registration method provided by the present disclosure can achieve ten-thousand-layer registration within only five seconds, meeting clinical real-time requirements. Moreover, the image registration method applies artificial intelligence deep learning algorithms for the extraction and segmentation of spinal segments, resulting in higher registration accuracy and efficiency, and an improved user experience.

According to an embodiment of the present disclosure, as shown in FIG. 9, the present disclosure further provides an image registration device 100 for a surgical robot, comprising: a capturing assembly 110, an acquisition assembly 120, and an information processing assembly 130. Wherein:

The capturing assembly 110 is configured to capture two-dimensional images of a surgical object and determine first guiding information in the two-dimensional images.

The acquisition assembly 120 is configured to acquire three-dimensional images of the surgical object and determine second guiding information in the three-dimensional images.

The information processing assembly 130 is configured to:
Calculating first pose information according to the first guiding information and the second guiding information;
Adjusting the three-dimensional images according to the first pose information, and acquire digitally reconstructed two-dimensional images in the adjusted three-dimensional images;
Calculating the similarity between the two-dimensional images and the digitally reconstructed two-dimensional images;
When the similarity meets a preset condition, project preoperative planning information onto the two-dimensional images according to the first pose information.

When the similarity does not meet the preset condition, update the first pose information to obtain second pose information, and adjust the three-dimensional images according to the second pose information.

The specific limitations of the image registration device 100 for a surgical robot are similar to the specific limitations of the image registration method 10 for a surgical robot described above. Reference may be made to the description of the image registration method 10 above, and will not be repeated herein.

According to an embodiment of the present disclosure, the present disclosure further provides an electronic device, comprising:
a processor; and
a memory storing a computer program which, when executed by the processor, causes the processor to perform the image registration method 10 for a surgical robot as described in one or more of the embodiments above.

According to an embodiment of the present disclosure, the present disclosure further provides a non-transitory computer-readable storage medium storing computer-readable instructions, which, when executed by a processor, cause the processor to perform the image registration method 10 for a surgical robot as described in one or more of the embodiments above.

Exemplarily, the digitally reconstructed radiographs (DRR) involved in the present disclosure refer to X-ray images generated from input CT images through an algorithm.

The PNP (Perspective-N-Point) algorithm involved in the present disclosure refers to a method for solving the external parameters of a camera (i.e., the pose of the camera) by using point correspondences consisting of N 3D spatial points and their corresponding 2D projection points, under known or unknown camera intrinsic parameters, by minimizing the reprojection error.

The above provides a detailed description of embodiments of the present application. Specific examples have been used herein to illustrate the principles and implementations of the present application. The description of the above embodiments is only intended to help understand the method and core idea of the present application. At the same time, any modifications or variations made by those skilled in the art based on the concept of the present application, within the scope of the specific embodiments and application range, fall within the protection scope of the present application. Therefore, the contents of this specification should not be understood as limiting the present application.

What is claimed is:

1. A method for image registration for a surgical robot, comprising:
capturing a two-dimensional image of a surgical object, and determining first guiding information in the two-dimensional image;
acquiring a three-dimensional image of the surgical object and preoperative planning information, and determining second guiding information in the three-dimensional image;
calculating first pose information according to the first guiding information and the second guiding information;
adjusting the three-dimensional image according to the first pose information, and acquiring a digitally reconstructed two-dimensional image in the adjusted three-dimensional image; and
calculating a similarity between the two-dimensional image and the digitally reconstructed two-dimensional image; wherein,
when the similarity meets a preset condition, projecting the preoperative planning information onto the two-dimensional image according to the first pose information; and
when the similarity does not meet the preset condition, updating the first pose information to obtain second pose information, and adjusting the three-dimensional image according to the second pose information.

2. The method according to claim 1, wherein the two-dimensional image comprises anteroposterior two-dimensional images and lateral two-dimensional images, the first guiding information comprises anteroposterior guiding information and lateral guiding information, and the capturing the two-dimensional images of the surgical object comprises:
capturing an anteroposterior two-dimensional image of the surgical object at a first position, and determining the anteroposterior guiding information in the anteroposterior two-dimensional image; and
capturing a lateral two-dimensional image of the surgical object at a second position, and determining the lateral guiding information in the lateral two-dimensional image;
wherein the anteroposterior guiding information comprises an anteroposterior center position information of the surgical object in the anteroposterior two-dimensional image and anteroposterior naming information, and the lateral guiding information comprises a lateral center position information of the surgical object in the lateral two-dimensional image and lateral naming information.

3. The method according to claim 1, wherein the acquiring the three-dimensional image of the surgical object and determining the second guiding information in the three-dimensional image comprises:
acquiring three-dimensional medical images of a patient's surgical region;
performing region of interest (ROI) cropping on the three-dimensional medical images;
performing single-segment segmentation and extraction on the cropped three-dimensional medical images;
selecting the surgical object from at least one segment and checking the segmentation result;
naming the surgical object; and
calculating a center position of the surgical object by a deep learning algorithm, and taking information of the naming and the center position of the surgical object as the second guiding information.

4. The method according to claim 1, wherein the two-dimensional image is captured by a two-dimensional image capturing device, and the calculating the first pose information according to the first guiding information and the second guiding information comprises:
acquiring scale information in the two-dimensional image;
acquiring internal parameters of the two-dimensional image capturing device;
calculating external parameters of the two-dimensional image capturing device according to the scale information and the internal parameters; and
obtaining the first pose information according to the external parameters and the first guiding information and the second guiding information.

5. The method according to claim 1, wherein the adjusting the three-dimensional image according to the first pose information and acquiring the digitally reconstructed two-dimensional image in the adjusted three-dimensional image comprises:
processing the three-dimensional image, simulating an X-ray source, calculating image density values in the ray direction, and obtaining the digitally reconstructed two-dimensional image.

6. The method according to claim 1, wherein the calculating the similarity between the two-dimensional image and the digitally reconstructed two-dimensional image comprises:
calculating the similarity using a normalized cross-correlation coefficient.

7. The method according to claim 6, wherein the calculating the similarity using a normalized cross-correlation coefficient comprises:
calculating a mean value and a standard deviation of the two-dimensional image and the digitally reconstructed two-dimensional image;
determining the normalized cross-correlation coefficient according to the mean value and the standard deviation; and
determining the similarity according to the normalized cross-correlation coefficient.

8. The method according to claim 7, wherein formulas for calculating the mean value and the standard deviation of the two-dimensional image and the digitally reconstructed two-dimensional image are as follows:
$$μ ( K ) = \frac{1}{\vert Ω \vert} {\sum }_{p ∈ Ω} K ( p )$$
$$σ ( K ) = \sqrt{\frac{1}{\vert Ω \vert - 1} {\sum }_{p ∈ Ω} {( K ( p ) - μ ( K ) )}^{2}}$$
where K is the two-dimensional image or the digitally reconstructed two-dimensional image, μ is the mean value, σ is the standard deviation, Ω is a total number of image pixels, and ρ is a pixel size of an image corresponding to the two-dimensional image K or the digitally reconstructed two-dimensional image K;
a formula for determining the normalized cross-correlation coefficient (NCC) according to the mean value and standard deviation is as follows:
$${S}_{N C C} ( I , J , {c}_{r} , {c}_{c} , r ) = - \frac{1}{\vert {Ω}_{{c}_{r} , {c}_{c} , r} \vert} {\sum }_{p ∈ {Ω}_{{c}_{r} , {c}_{c} , r}} \frac{( I ( p ) - {μ}_{I} ) ( J ( p ) - {μ}_{J} )}{{σ}_{I} {σ}_{J}}$$
where I is the two-dimensional image, J is the digitally reconstructed two-dimensional image, cᵣ is a coordinate of a center position in the two-dimensional image, c_{c} is a coordinate of a center position in the digitally reconstructed two-dimensional image, r is half of a side length of the two-dimensional image and the digitally reconstructed two-dimensional image;
a formula for determining the similarity according to the normalized cross-correlation coefficient is as follows:
$${s}_{G N C C} ( I , J , {c}_{{r}_{1}} , {c}_{{c}_{1}} , {r}_{1} ) = {S}_{N C C} ( {∇}_{x} I , {∇}_{x} J , {c}_{{r}_{1}} , {c}_{{c}_{1}} , {r}_{1} ) + {S}_{N C C} ( {∇}_{y} I , {∇}_{y} J , {c}_{{r}_{1}} , {c}_{{c}_{1}} , {r}_{1} )$$
where ∇ₓI is an X-gradient image of the two-dimensional image, ∇_{y}I is a Y-gradient image of the two-dimensional image, ∇ₓJ is an X-gradient image of the two-dimensional image, ∇_{y}J is a Y-gradient image of the digitally reconstructed two-dimensional image, S_{GNCC} is a sum of the NCCs of X-gradient and Y-gradient gradient images, Cᵣ_{₁} is a coordinate of a center position in the X-gradient images, c_{c}_{₁} is a coordinate of a center position in the Y-gradient images, r₁ is half of a side length of the X-gradient images and the Y-gradient images, and S_{GNCC}(I, J, cᵣ_{₁}, c_{c}_{₁}, r₁) is the similarity of the X-gradient images in a square region with cᵣ_{₁} as the center and 2r₁ as a side length and the Y-gradient images in a square region with c_{c}_{₁} as the center and 2r₁ as a side length.

9. The method according to claim 1, wherein the projecting the preoperative planning information onto the two-dimensional image according to the first pose information comprises:
projecting preoperatively planned pedicle screw positions onto the two-dimensional image.

10. The method according to claim 1, wherein the updating the first pose information to obtain the second pose information comprises:
using the first pose information as an initial value, and updating the first pose information using a gradient-independent optimization algorithm to obtain the second pose information.

11. An image registration device for a surgical robot, comprising:
a capturing assembly configured to capture two-dimensional images of a surgical object and determine first guiding information in the two-dimensional images;
an acquisition assembly configured to acquire three-dimensional images of the surgical object and determine second guiding information in the three-dimensional images; and
an information processing assembly configured to:
calculate first pose information according to the first guiding information and the second guiding information;
adjust the three-dimensional image according to the first pose information, and acquire a digitally reconstructed two-dimensional image in the adjusted three-dimensional image; and
calculate similarity between the two-dimensional image and the digitally reconstructed two-dimensional image; wherein,
when the similarity meets a preset condition, project preoperative planning information onto the two-dimensional image according to the first pose information; and
when the similarity does not meet the preset condition, update the first pose information to obtain second pose information, and adjust the three-dimensional image according to the second pose information.

12. An electronic device, comprising:
a processor; and
a memory storing a computer program, which, when executed by the processor, causes the processor to perform the method according to claim 1.

13. A non-transitory computer-readable storage medium storing computer-readable instructions, wherein when the instructions are executed by a processor, the processor performs the method according to claim 1.
